(19) United States
(12) Patent Application Publication
(10) Pub. No.: US 2020/0187907 A1
(43) Pub. Date: Jun. 18, 2020

(54) ULTRASONIC TRANSDUCER AND MANUFACTURING METHOD THEREFOR

(71) Applicant: SHENZHEN INSTITUTES OF ADVANCED TECHNOLOGY CHINESE ACADEMY OF SCIENCES, Shenzhen (CN)

(72) Inventors: Weibao Qiu, Shenzhen (CN); Wu Sun, Shenzhen (CN); Min Su, Shenzhen (CN); Suzi Liang, Shenzhen (CN); Zhiqiang Zhang, Shenzhen (CN); Hairong Zheng, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 16/607,839

(22) PCT Filed: Jul. 26, 2018
(86) PCT No.: PCT/CN2018/097184

§ 371 Date: Oct. 24, 2019

(30) Foreign Application Priority Data
Mar. 28, 2018 (CN) 201810265439.5

(51) Int. Cl. A61B 8/00; B60B 3/04; B06B 1/06

(52) U.S. Cl. CPC A61B 8/4494; B06B 1/0614; B06B 1/0629; B60B 3/04

(57) ABSTRACT

Provided is an ultrasonic transducer and a preparation method thereof. The ultrasonic transducer includes a housing. A piezoelectric layer is disposed in the housing and includes at least two piezoelectric array elements. A frequency interval between the piezoelectric array elements is 50 kHz to 1.2 MHz. An acoustic lens is disposed at a front end of the piezoelectric layer and is used for ensuring that the piezoelectric array elements having different frequencies have a common focus.

### CROSS-REFERENCE TO RELATED APPLICATIONS

This is a National Stage Application, filed under 35 U.S.C. 371, of International Patent Application No. PCT/CN2018/097184, filed on Jul. 26, 2018, which claims priority to Chinese patent application No. 201810265439.5 filed on Mar. 28, 2018, contents of both of which are incorporated herein by reference in their entireties.

### TECHNICAL FIELD

The present disclosure relates to the technical field of medical ultrasonics and, in particular, to an ultrasonic transducer and a preparation method thereof.

### BACKGROUND

The medical ultrasonics technology has been widely used in clinical diagnosis and treatment. Ultrasound diagnosis mainly uses the ultrasound echo to obtain imaging information of the tissue, and provides clinicians with the necessary diagnostic reference. Ultrasound treatment uses the mechanical effects, thermal effects, and cavitation effects of ultrasonic waves for the treatment of diseases. Specifically, it can be divided into the high-dose ultrasonic thermal ablation technology and the low-dose ultrasonic modulation technology. High intensity focused ultrasound (HIFU) is a typical ultrasonic thermal ablation technology. HIFU can penetrate the tissue, reach the target area, destroy the tumor in the body, and finally absorb the destroyed tumor through the immune system of the body, so as to achieve the efficacy of non-invasive treatment. The low-dose ultrasound treatment mainly includes: ultrasonic vascular thrombolysis, ultrasound-induced blood-brain barrier opening, and ultrasound neuromodulation. The so-called ultrasonic vascular thrombolysis is to use the ultrasonic wave to destroy and dredge blood spots in order to achieve the purpose of treatment. The blood-brain barrier is a barrier, between blood vessels and the brain, used for selectively blocking certain substances from entering the brain. This is usually beneficial for the protection of the body. However, the blood-brain barrier also weakens the treatment effect of the drug on the patient. Focused ultrasound can temporarily remove the blood-brain barrier and allow the drug to pass through the barrier to the brain, effectively improving the treatment effect of the drug. Ultrasound neuromodulation is to stimulate the nerve by ultrasound, make the nervous system excited or inhibited, regulate the nerve activity of the organism, and change the response of the neural circuit, thereby contributing to the treatment of neuropsychiatric diseases. Ultrasound neuromodulation becomes an effective means through human intervention in the neural circuits of living organisms and then the study of brain functions (such as cognition, feeling).

The performance of the ultrasonic transducer, as a key component of the ultrasound neuromodulation, plays a decisive role to the treatment effect. Currently, it has been reported in existing literature that single-frequency, dual-frequency excitation single-element ultrasonic transducers are used for ultrasonic thrombolysis. In fact, with the limited transducer bandwidth, when the single-element ultrasonic transducer is excited by a dual-frequency signal or a multi-frequency signal, the electroacoustic conversion efficiency will be reduced at the non-resonant frequency point, and the impedance matching circuit also needs to meet higher requirements. In use, in order to achieve a better effect, power compensation is necessary for the non-resonant frequency excitation signal.

Neurological diseases have always been a challenge in the medical field, and traditional chemical drugs have been difficult to work with. The neuromodulation is gaining more and more attention. For cardiovascular diseases, the traditional surgical interventional treatment means requires implantation of catheters in the blood vessels, with a risk of hemorrhoea. The ultrasound-induced treatment method receives special attention due to the noninvasion, high-resolution and other advantages. Several main methods based on ultrasound treatment that are proposed at present in the field are described below.

An ultrasound treatment method based on the single-element transducer is included. This method is simple to implement. The single-element transducer may be formed by connecting the signal generator, the signal amplifier and other devices existing in the market. Or a dedicated device may be customized according to requirements and is used with the single-element ultrasonic transducer. In terms of stimulation detection, a method based on detecting a bioelectric signal or a method of magnetic resonance imaging (MRI) imaging may usually be combined.

An ultrasonic treatment method based on single excitation frequency phased array transducer is included. A French company, Image Guided Therapy, designs and develops a phased array ultrasonic neuromodulation device based on a single sinusoidal excitation frequency. Currently, the device may achieve phased array electronic focusing of 128 array elements, and is combined with MRI imaging guidance to be applied to HIFU, neuromodulation and the like. Currently in these common methods, not too many designs are provided for the transducer, and typically a single-frequency single-element or an array ultrasonic transducer is used.

A US invention patent entitled “Dual-frequency ultrasound transducer” (Patent Publication No.: US20120267986A1) designs a dual-frequency ultrasonic transducer that can support a low frequency (100 kHz) and a high frequency (1 to 3 MHz). When the transducer is excited by the voltage of the low frequency oscillation component, low frequency resonance occurs; and when the transducer is excited by the voltage of the high frequency oscillation component, high frequency resonance occurs. The transducer can enhance the penetration depth of the ultrasound. However, the transducer cannot improve the cavitation effects of the ultrasound without increasing the power loss of the excitation system. At present, the transducer is mainly used in the field of medical beauty for increasing the penetrability of the skin, reducing the barrier effect of the outer stratum corneum of the skin, and improving the effect of skin cosmetic treatment, and is not suitable for applications such as neuromodulation, blood-brain barrier opening, ultrasound administration and vascular thrombolysis.

In traditional ultrasound treatment applications, the single-frequency single-element or array ultrasonic transducer is used. In consideration of the limited bandwidth of the transducer, larger energy loss is caused by directly using the dual-frequency or multi-frequency excitation signal to drive the ultrasonic transducer, which is not conducive to maximizing the performance of the transducer. In addition, since the excitation signal includes multiple frequency components, higher requirements are imposed on the impedance matching circuit of the excitation system, and a risk that the reflected signal power is large exists.

A Chinese invention patent entitled “a dual-frequency double-layer power-enhanced ring-shaped high-intensity focused ultrasound transducer” (Patent Publication No.: CN201510169324.2) discloses a ring-shaped high-intensity focused ultrasound transducer. The inner ring is a high-frequency piezoelectric wafer, the outer ring is a low-frequency piezoelectric wafer, and the two layers of piezoelectric wafers have annular concave self-focusing structures. The high-frequency piezoelectric wafers are on the same spherical surface and are confocal. Due to the large difference in frequency and the difference in thickness of the wafer, the two layers of annular concave wafer nesting structure is difficult to ensure that the two wafers are on the same spherical surface and then that the focus points of the two wafers are at the same position in the actual preparation process of the transducer.

### SUMMARY

In view of the deficiencies of the related art described above, an ultrasonic transducer with dual frequency and multiple frequencies integrated is designed in the present disclosure. The transducer may simultaneously receive two or more frequency component excitation signals. The two frequencies may be relatively close. The use of different excitation sequences allows to enhance the cavitation effect of ultrasound without increasing power loss. The present disclosure uses two or more piezoelectric array elements having different frequencies to form ultrasonic transducers of two or more frequencies, and the piezoelectric array elements having different frequencies are focused at the same focus through the acoustic lens. The structure is easy to prepare and low in cost. It has been experimentally verified that the dual-frequency and multi-frequency integrated ultrasonic transducer can enhance the treatment effect, enhance the feasibility, and achieve better treatment effects in applications such as blood-brain barrier opening and ultrasonic thrombolysis.

The present disclosure provides an ultrasonic transducer including a housing. A piezoelectric layer is disposed in the housing and includes at least two piezoelectric array elements having different frequencies. A frequency interval between the piezoelectric array elements is 50 kHz to 1.2 MHz.

In an embodiment, the piezoelectric layer includes a low-frequency piezoelectric array element and a high-frequency piezoelectric array element. The frequency interval between the low-frequency piezoelectric array element and the high-frequency piezoelectric array element is 50 kHz to 1.2 MHz.

In an embodiment, the piezoelectric layer includes a low-frequency piezoelectric array element, an intermediate-frequency piezoelectric array element and a high-frequency piezoelectric array element. The frequency interval among the low-frequency piezoelectric array element, the intermediate-frequency piezoelectric array element and the high-frequency piezoelectric array element is 50 kHz to 1.2 MHz.

In an embodiment, the piezoelectric array elements are planar or concave.

In an embodiment, the piezoelectric array elements are arranged symmetrically with respect to a circular center axis or arranged in a linear array.

In an embodiment, an axial cross section of the piezoelectric layer is circular, triangular or square.

In an embodiment, at least one matching layer is disposed on a front end of the piezoelectric layer, and an acoustic lens is disposed on a front end of the matching layer and is used for ensuring that the piezoelectric array elements having different frequencies have a common focus. A backing layer is disposed on a back end of the piezoelectric layer. Electrodes on upper and lower surfaces of each of the piezoelectric elements of the piezoelectric layer are respectively connected to a positive electrode and a negative electrode of a cable.

The present disclosure further provides an ultrasonic transducer preparation method. The method includes steps described below.

In a step S1, side surfaces of at least two piezoelectric array elements are bonded to form a piezoelectric layer.

In a step S2, electrodes are deposited on upper and lower surfaces of each of the piezoelectric array elements, and the electrodes are connected to a positive electrode and a negative electrode of a cable.

In a step S3, the piezoelectric layer connected to the cable is fixed to an inner side of a housing, and a backing material is deposited on a lower surface of the piezoelectric layer to form a backing layer fixed to the inner side of the housing.

In a step S4, a matching material is deposited on an upper surface of the piezoelectric layer to form a matching layer fixed to the inner side of the housing.

In a step S5, a layer of acoustic lens is formed over an upper surface of the matching layer. The acoustic lens is used for ensuring that the piezoelectric array elements having different frequencies have a common focus. Propagation time of a sound wave of each of the piezoelectric array elements is calculated through a radius of curvature and a speed of sound of the layer of acoustic lens, and excitation time of the each of the piezoelectric array elements is adjusted to overlap focuses of the piezoelectric array elements.

In an embodiment, in the step S2, the electrodes are deposited on the upper and lower surfaces of each of the piezoelectric array elements to form an array element positive electrode and an array element negative electrode. The array element positive electrode is connected to the positive electrode of the cable. The array element negative electrode is connected to the negative electrode of the cable, or the array element negative electrode is connected to a metal housing via a conductive material and the metal housing is connected to the negative electrode of the cable.

In an embodiment, the backing material is epoxide resin with filler, and the matching material is the epoxide resin with the filler.

The beneficial effects of implementing the present disclosure are mainly as follows.

1) An ultrasonic transducer is provided to support simultaneous excitation of dual-frequency and multi-frequency signals while maintaining high electro-acoustic conversion efficiency. The dual-frequency or multi-frequency combination transducers can improve the frequency bandwidth of the ultrasonic transducer, reduce the design difficulty of the broadband impedance matching circuit, and facilitate the transmission and reception of ultrasonic signals and post-processing.

2) The combination of planar piezoelectric array elements having two or more different frequencies and the use of focusing acoustic lens allow to, compared with the concave wafer nesting structure, more easily ensure the common focus, and reduce the preparation difficulty of the ultrasonic transducer.

3) The cavitation effect of ultrasound can be improved without increasing the power loss of the excitation system.

4) Different frequency combinations excite the ultrasonic transducer in cooperation with the ultrasound excitation system and in use of different excitation sequences can significantly improve the treatment effect in applications such as neuromodulation, blood-brain barrier opening, ultrasound administration, vascular thrombolysis.

### BRIEF DESCRIPTION OF DRAWINGS

For a better understanding of the present disclosure, reference may be made to the following drawings for illustrating the related art or the embodiments. The drawings will briefly show a part of products or methods involved in the embodiments or in the related art. The basic information of the drawings is as follows.

FIG. 1 is a schematic diagram of an ultrasonic transducer according to an embodiment;

FIG. 2 is a cross-sectional view of an ultrasonic transducer according to an embodiment;

FIG. 3 is an exploded view of an ultrasonic transducer according to an embodiment;

FIG. 4 is an arrangement diagram of piezoelectric array elements having two frequencies according to an embodiment; and

FIG. 5 is an arrangement diagram of piezoelectric array elements having three frequencies according to an embodiment.

1—Matching layer, 2—Acoustic lens, 3—Piezoelectric layer, 31—Low-frequency piezoelectric array element, 32—High-frequency piezoelectric array element, 33—Intermediate-frequency piezoelectric array element, 4—Backing layer, 5—Housing, 6—Cable.

### DETAILED DESCRIPTION

The present disclosure will be further described. It is obvious that the described embodiments are only part, not all, of embodiments of the present disclosure.

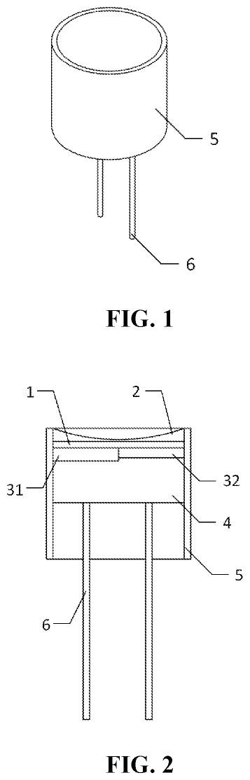

As shown in FIG. 1 to FIG. 3, the present example provides an ultrasonic transducer including a housing 5. A piezoelectric layer 3 is disposed in the housing 5 and includes at least two piezoelectric array elements having different frequencies, and a frequency interval between the piezoelectric array elements is 50 kHz to 1.2 MHz. In another embodiment, the frequency interval between the piezoelectric array elements may be 50 kHz to 1 MHz; and the frequency interval between the piezoelectric array elements may further be 50 kHz to 0.8 MHz. The piezoelectric array elements are concave. In another embodiment, the piezoelectric array elements may be planar. Compared with the concave wafer nesting structure in the related art, in the embodiment, the combination of planar piezoelectric array elements having two or more different frequencies and the use of focusing acoustic lens can more easily ensure the common focus and reduce the preparation difficulty of the ultrasonic transducer. The piezoelectric array elements are arranged symmetrically with respect to a circular center axis. In another embodiment, the piezoelectric array elements may be arranged in a linear array. An axial cross section of the piezoelectric layer 3 is circular. In another embodiment, the axial cross section of the piezoelectric layer 3 may be triangular or square. Correspondingly, the housing 5 may be configured to be circular, triangular or square.

Specifically, the ultrasonic transducer includes the housing 5. The piezoelectric layer 3 is disposed in the housing 5. The piezoelectric layer includes a low-frequency piezoelectric array element 31 and a high-frequency piezoelectric array element 32. The frequency interval between the low-frequency piezoelectric array element 31 and the high-frequency piezoelectric array element 32 is 50 kHz to 1.2 MHz. An acoustic lens 2 is disposed on the piezoelectric layer 3 and is used for ensuring that the low-frequency piezoelectric array element 31 and the high-frequency piezoelectric array element 32 have a common focus. In an embodiment, the focal length is 5 cm to 10 cm. In the embodiment, the frequency of each of the low-frequency piezoelectric array element 31 and the high-frequency piezoelectric array element 32 is 0.5 MHz to 2 MHz.

In the embodiment, at least one matching layer 1 is disposed on a front end of the piezoelectric layer 3, and has a main function of improving the sound propagation efficiency of the transducer. One or more matching layers are provided. The acoustic lens 2 is disposed on a front end of the matching layer. A backing layer 4 is disposed on a back end of the piezoelectric layer 3.

Electrodes on upper and lower surfaces of each of the piezoelectric elements of the piezoelectric layer 3 are respectively connected to a core wire and a ground wire of a cable 6. Each piezoelectric array element of the ultrasonic transducer is led out through a coaxial cable. In an embodiment, the ultrasonic transducer is a dual-frequency transducer, includes a low-frequency piezoelectric array element 31 and a high-frequency piezoelectric array element 32, and therefore has two coaxial cables. The core wire and the negative ground wire of each coaxial cable are connected to upper and lower surfaces of respective one of the low-frequency piezoelectric array element 31 and the high-frequency piezoelectric array element 32. Specifically, the positive electrodes of the two cables 6 are connected to the respective positive electrodes of the two piezoelectric array elements, and the negative electrodes of the two cables 6 are connected to the respective negative electrodes of the two piezoelectric array elements.

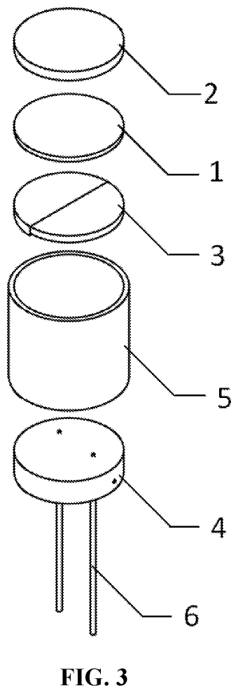

Specifically, as shown in FIG. 2 and FIG. 3, a matching layer 1 is disposed on the upper surface of the piezoelectric layer 3. The acoustic lens 2 is disposed over the upper surface of the matching layer 1 and is used for ensuring that the piezoelectric array elements having different frequencies have a common focus. The backing layer 4 is disposed on the lower surface of the piezoelectric layer 3. Electrodes on upper and lower surfaces of each of the piezoelectric elements of the piezoelectric layer 3 are respectively connected to a positive electrode and a negative electrode of the cable 6. One end of the cable 6 is connected to the piezoelectric layer 3, and the other end of the cable 6 passes through the backing layer 4 and extends to the outside of the housing 5.

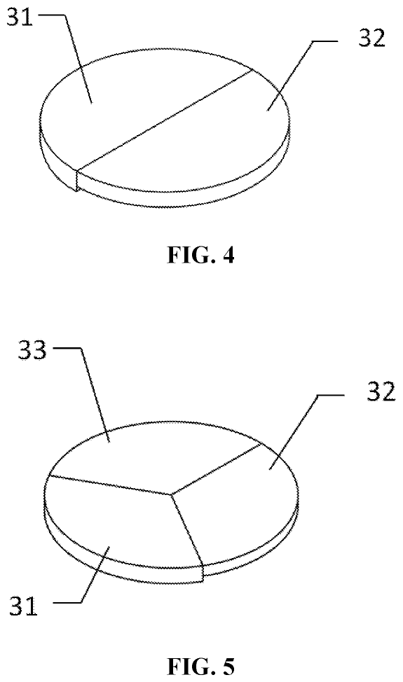

In the embodiment, the piezoelectric layer 3 includes the low-frequency piezoelectric array element 31 and the high-frequency piezoelectric array element 32. The frequency interval between the low-frequency piezoelectric array element 31 and the high-frequency piezoelectric array element 32 is 50 kHz to 1.2 MHz. The high frequency and the low frequency are relative, and the difference between the high frequency and the low frequency may be small. As shown in FIG. 4, the ultrasonic transducer is a dual-frequency ultrasonic transducer, includes two semi-circular piezoelectric array elements having different frequencies. The two semi-circular piezoelectric array elements form a circular piezoelectric layer 3. The low-frequency piezoelectric array element 31 has a frequency of 650 kHz, and the high-frequency piezoelectric array element 32 has a frequency of 1 MHz. Two piezoelectric array elements having different frequencies are combined in one plane, and the sound field of the dual-frequency transducer is focused to the same position through the acoustic lens 2, thereby improving the sound field intensity at the focus position.

In the related art, a technical scheme of stacking up and down piezoelectric elements having different frequencies is often used; while in the embodiment, different piezoelectric wafers are ensured to have the common focus through the combination of multiple piezoelectric wafers having different frequencies and the use of the acoustic lens. In practical applications, the low-frequency piezoelectric array element 31 and the high-frequency piezoelectric array element 32 may not be arranged in the same plane, and the combination manner of the piezoelectric array elements may be specifically designed according to the design of the acoustic lens.

In the ultrasonic thrombolysis application, the 650 kHz sinusoidal signal and the 1 MHz sinusoidal signal are respectively used and combined to excite the dual-frequency ultrasonic transducer of the embodiment. As a control group, a single-frequency group uses the 650 kHz sinusoidal signal or the 1 MHz sinusoidal signal to excite a common ultrasonic transducer. The parameters of the dual-frequency group and the single-frequency group are respectively configured, and the parameters such as the action time, the pulse repetition frequency, the duty ratio of the excitation signal, the power are configured to be the same.

The experimental results show that the dual-frequency stimulation can reduce the cavitation threshold of ultrasound thrombolysis in the application of ultrasound thrombolysis, achieving the thrombolysis efficiency double that of the single-frequency case. In other words, on the premise of the same thrombolysis efficiency, the treatment time of the dual-frequency thrombolysis can be shortened to half of the treatment time of single-frequency thrombolysis. The dual-frequency ultrasonic transducer can increase the sound pressure generated by the common ultrasonic transducer by 30%, which can further reduce the energy for exciting the ultrasonic transducer device. This can reduce the accumulation of heat and reduce the risk of heat build-up for the transcranial ultrasound application. Compared with the related art, in the embodiment, the difference between the high frequency and the low frequency is small, the cavitation effect of the ultrasound can be improved without increasing the power loss of the excitation system, and the thrombolysis effect is good.

In an embodiment, the piezoelectric layer 3 includes the low-frequency piezoelectric array element 31, an intermediate-frequency piezoelectric array element 33 and the high-frequency piezoelectric array element 32. The frequency interval among the low-frequency piezoelectric array element 31, the intermediate-frequency piezoelectric array element 33 and the high-frequency piezoelectric array element 32 is 50 kHz to 1.2 MHz. The high frequency, the intermediate frequency and the low frequency are relative; and the difference among the high frequency, the intermediate frequency and the low frequency may be small. In the embodiment, the frequency of each of the low-frequency piezoelectric array element 31, the intermediate-frequency piezoelectric array element 33 and the high-frequency piezoelectric array element 32 is 0.5 MHz to 2 MHz. As shown in FIG. 5, the ultrasonic transducer is a triple-frequency ultrasonic transducer, includes three sector-shaped piezoelectric array elements having different frequencies. The three sector-shaped piezoelectric array elements form a circular piezoelectric layer 3, and may be the same or different. In practical applications, the low-frequency piezoelectric array element 31, the intermediate-frequency piezoelectric array element 33 and the high-frequency piezoelectric array element 32 may be disposed in the same plane or not; and the combination manner of the piezoelectric array elements may be specifically designed according to the design of the acoustic lens. The low-frequency piezoelectric array element 31 has a frequency of 1.4 MHz, the intermediate-frequency piezoelectric array element 33 has a frequency of 1.45 MHz, and the high-frequency piezoelectric array element 32 has a frequency of 1.5 MHz. It has been experimentally verified that three frequencies combined in use for excitation can slightly (5%) improve the efficiency compared with dual frequencies in the application of ultrasonic thrombolysis. Compared with the related art, in the embodiment, the difference among the high frequency, the intermediate frequency and the low frequency is small, the cavitation effect of the ultrasound can be improved without increasing the power loss of the excitation system, and the thrombolysis effect is good. In practical applications, the piezoelectric elements are not limited to having two or three different frequencies, and can be set according to actual needs. In the embodiment, piezoelectric array elements having different frequencies are combined to form the ultrasonic transducer having two or more frequencies, and piezoelectric array elements having different frequencies are combined to produce mixed-frequency ultrasound. The dual-frequency or multi-frequency combination transducers can improve the frequency bandwidth of the ultrasonic transducer, reduce the design difficulty of the broadband impedance matching circuit, and facilitate the transmission and reception of ultrasonic signals and post-processing. Two or more piezoelectric array elements having different frequencies are combined in one plane, and the sound field of each of the dual-frequency transducer and the multi-frequency transducer is focused to the same position through the acoustic lens 2, thereby improving the sound field intensity at the focus position.

The embodiment further provides an ultrasonic transducer preparation method. The method includes steps described below.

In a step S1, side surfaces of the low-frequency piezoelectric array element 31 and the high-frequency piezoelectric array element 32 are bonded to form the piezoelectric layer 3. Specifically, the low-frequency piezoelectric array element 31 and the high-frequency piezoelectric array element 32 are bonded via epoxide resin or other bonding materials.

In a step S2, electrodes are deposited on upper and lower surfaces of each of the piezoelectric array elements, and the electrodes are connected to the positive electrode and the negative electrode of the cable 6.

Specifically, in the step S2, the electrodes are deposited on the upper and lower surfaces of each of the piezoelectric array elements to form an array element positive electrode and an array element negative electrode. Each array element positive electrode is connected to the positive electrode (core wire) of the cable 6. In the embodiment, the cable 6 is a coaxial cable. Each piezoelectric array element of the ultrasonic transducer is led out through a respective coaxial cable. The negative electrode of the transducer is connected in two manners: each array element negative electrode is connected to the negative electrode (ground wire) of a respective coaxial cable; or each array element negative electrode is connected to the metal housing 5 via a conductive material, and the metal housing 5 is connected to the negative electrode (ground wire) of each coaxial cable. In an embodiment, the conductive material is conductive silver paste.

In a step S3, the piezoelectric layer 3 connected to the cable 6 is fixed to an inner side of the housing 5, and a backing material is deposited on a lower surface of the piezoelectric layer 3 to form the backing layer 4 fixed to the inner side of the housing 5. In an embodiment, the backing material is epoxide resin with filler.

In a step S4, a matching material is deposited on an upper surface of the piezoelectric layer 3 to form the matching layer 1 fixed to the inner side of the housing 5. In an embodiment, the matching material is the epoxide resin with the filler.

In a step S5, a layer of acoustic lens 2 is formed at a front end of the matching layer 1. The acoustic lens is used for ensuring that the piezoelectric array elements having different frequencies have a common focus. The propagation time of a sound wave is calculated through a radius of curvature and a speed of sound of the layer of acoustic lens 2, and excitation time is adjusted to have focuses of the piezoelectric array elements overlapped.

Specifically, a layer of acoustic lens 2 is formed over an upper surface of the matching layer 1. The acoustic lens is used for ensuring that the piezoelectric array elements having different frequencies have a common focus. The propagation time of a sound wave of each of the piezoelectric array elements is calculated through a radius of curvature and a speed of sound of the layer of acoustic lens 2, and excitation time of the each of the piezoelectric array elements is adjusted to overlap focuses of the piezoelectric array elements. Compared with the focusing through concave wafer nesting in the related art, in the embodiment, the combination of planar piezoelectric array elements having two or more different frequencies and the use of the focusing acoustic lens 2 can reduce the preparation difficulty.

What is claimed is:

1. An ultrasonic transducer, comprising a housing, wherein a piezoelectric layer is disposed in the housing and comprises at least two piezoelectric array elements having different frequencies, and a frequency interval between the piezoelectric array elements is 50 kHz to 1.2 MHz.

2. The ultrasonic transducer of claim 1, wherein the piezoelectric layer comprises a low-frequency piezoelectric array element and a high-frequency piezoelectric array element, and the frequency interval between the low-frequency piezoelectric array element and the high-frequency piezoelectric array element is 50 kHz to 1.2 MHz.

3. The ultrasonic transducer of claim 1, wherein the piezoelectric layer comprises a low-frequency piezoelectric array element, an intermediate-frequency piezoelectric array element and a high-frequency piezoelectric array element, and the frequency interval among the low-frequency piezoelectric array element, the intermediate-frequency piezoelectric array element and the high-frequency piezoelectric array element is 50 kHz to 1.2 MHz.

4. The ultrasonic transducer of claim 1, wherein the piezoelectric array elements are planar or concave.

5. The ultrasonic transducer of claim 1, wherein the piezoelectric array elements are arranged symmetrically with respect to a circular center axis or arranged in a linear array.

6. The ultrasonic transducer of claim 1, wherein an axial cross section of the piezoelectric layer is circular, triangular or square.

7. The ultrasonic transducer of claim 1, wherein at least one matching layer is disposed on a front end of the piezoelectric layer, and an acoustic lens is disposed on a front end of the matching layer and is used for ensuring that the piezoelectric array elements having different frequencies have a common focus.

8. The ultrasonic transducer of claim 1, wherein a backing layer is disposed on a back end of the piezoelectric layer; and electrodes on upper and lower surfaces of each of the piezoelectric elements of the piezoelectric layer are respectively connected to a positive electrode and a negative electrode of a cable.

9. An ultrasonic transducer preparation method, comprising:
step S1: bonding side surfaces of at least two piezoelectric array elements to form a piezoelectric layer;
step S2: depositing electrodes on upper and lower surfaces of each of the piezoelectric array elements, and connecting the electrodes to a positive electrode and a negative electrode of a cable;
step S3: fixing the piezoelectric layer connected to the cable to an inner side of a housing, and depositing a backing material on a lower surface of the piezoelectric layer to form a backing layer fixed to the inner side of the housing;
step S4: depositing a matching material on an upper surface of the piezoelectric layer to form a matching layer fixed to the inner side of the housing; and
step S5: forming a layer of acoustic lens over an upper surface of the matching layer, wherein the acoustic lens is used for ensuring that the piezoelectric array elements having different frequencies have a common focus, propagation time of a sound wave of each of the piezoelectric array elements is calculated through a radius of curvature and a speed of sound of the layer of acoustic lens, and excitation time of the each of the piezoelectric array elements is adjusted to overlap focuses of the piezoelectric array elements.

10. The ultrasonic transducer preparation method of claim 9, wherein in the step S2, the electrodes are deposited on the upper and lower surfaces of each of the piezoelectric array elements to form an array element positive electrode and an array element negative electrode, wherein the array element positive electrode is connected to the positive electrode of the cable; the array element negative electrode is connected to the negative electrode of the cable, or the array element negative electrode is connected to a metal housing via a conductive material and the metal housing is connected to the negative electrode of the cable.

11. The ultrasonic transducer preparation method of claim 10, wherein the backing material is epoxide resin with filler, and the matching material is the epoxide resin with the filler
